(19) United States
(12) Patent Application Publication
(10) Pub. No.: US 2004/0142853 A1
(43) Pub. Date: Jul. 22, 2004

(54) Stimulation of hair growth by compositions containing peptide copper complexes and minoxidil

(75) Inventor: Leonard M. Patt, Seattle, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: ProCyte Corporation, Redmond, WA

(21) Appl. No.: 10/696,506

(22) Filed: Oct. 29, 2003

Related U.S. Application Data
(60) Provisional application No. 60/424,550, filed on Nov. 7, 2002.

(51) Int. Cl. A61K 38/16

(52) U.S. Cl. 514/6; 530/400

(57) ABSTRACT

Disclosed are compositions, useful for stimulating hair growth in warm-blooded animals in need thereof, that combine at least one peptide copper complex and Minoxidil. The disclosed compositions may be formulated so as to be suitable for topical application or for intradermal injection to areas of skin in need thereof. Also disclosed are methods for stimulating hair growth to thereby treat various hair growth afflictions, such as androgenetic alopecia, alopecia areata, or secondary alopecia. Generally, the disclosed methods comprise applying, topically or via intradermal injection, a stimulatory effective amount of a disclosed composition to an area of a patient's skin in need thereof. In one embodiment, the disclosed method employs such application in a continued and periodic fashion to arrest and/or reverse androgenetic alopecia.

### CROSS-REFERENCE TO RELATED APPLICATION

This application claims the benefit of U.S. Provisional Patent Application No. 60/424,550 filed Nov. 7, 2002, which provisional application is incorporated herein by reference in its entirety.

### BACKGROUND OF THE INVENTION

1. Field of the Invention

The present invention generally relates to chemical compositions useful for stimulating hair growth and, more specifically, to compositions containing a peptide copper complex and Minoxidil.

2. Description of the Related Art

Hair loss is a common human affliction. Particularly common is androgenetic alopecia (AGA), which is characterized by loss of scalp hair among males and females as they age (i.e., male pattern baldness and female pattern baldness). Other hair loss afflictions include alopecia areata (AA), which refers to microscopically inflammatory, usually reversible, patchy loss of hair, and hair loss associated with chemotherapy or radiation treatment (i.e., secondary alopecia).

Hair is normally divided into two types, namely, “terminal hairs” and “vellus hairs.” Terminal hairs are long hairs that are coarse and pigmented, and that grow from follicles developed deep within the dermis. Vellus hairs are typically thin, non-pigmented hairs that grow from smaller hair follicles located superficially in the dermis. As alopecia progresses, there is a change from terminal to vellus type hair associated with a corresponding diminishment of the hair follicles.

Also contributing to alopecia is an alteration in the growth cycle of hair. Hair typically progresses through three cycles: anagen (active hair growth), catagen (transition phase), and telogen (resting phase during which the hair shaft is shed prior to new growth). Normally, about 88% of the hairs on the scalp are in the anagen phase, with only about 1% being in the catagen phase and the remainder in the telogen phase. As baldness develops, a progressively greater proportion of the hairs are in the telogen and a correspondingly lesser proportion are in the active growth anagen phase.

Further associated with alopecia is a significant decrease in both the size and density of hair follicles. For example, it has been reported that bald human subjects ranging in age from 30 to 90 years have, on the average, only about 306 follicles per square centimeter. This represents about 33% less than the average of about 460 follicles per square centimeter for non-bald subjects in the same age range. It is a combination of the above factors that produces baldness.

A variety of procedures and drugs have been utilized in attempting to treat hair loss. One common technique involves hair transplantation. Briefly, the technique transplants plugs of hair-containing skin from areas of the scalp where hair is growing to bald or balding areas of the scalp. This approach, however, is costly, time-consuming and quite painful. It is also inadequate in that it restores only a very small fraction of the hair missing from a normal, healthy head of hair.

Other non-drug solutions to this problem include, for example, ultra-violet radiation, massage, psychiatric treatment, revascularization surgery, acupuncture and exercise therapy. However, none of these solutions has been generally accepted as being effective.

Drug therapy has been the most common approach to solving the problem of AGA. A variety of drugs ranging from vitamins to hormones have been tried with very limited success. Greater activity has been realized with the use of a hair-growth agent sold under the tradename “Minoxidil,” disclosed in U.S. Pat. Nos. 3,461,461 and 4,596,812 assigned to Upjohn. In addition, certain peptide copper complexes have proven to have activity as hair-growth agents. For example, U.S. Pat. Nos. 5,177,061; 5,120,831; 5,214,032; 5,538,945; 5,550,183 and 6,017,888 disclose certain peptide copper complexes that have exhibited activity in stimulating the growth of hair in warm-blooded animals.

While progress has, thereby, been made in stimulating hair-growth through drug therapy, there remains a need in the art for compositions that provide a stimulatory effect on hair growth greater than that achieved thus far. The present invention fulfills this need and provides further related advantages.

### BRIEF SUMMARY OF THE INVENTION

In brief, the present invention is directed to compositions having utility for stimulating hair growth in warm-blooded animals so as to, for example, arrest and/or reverse hair loss. A “warm-blooded animal,” as that expression is used herein, includes a human, and is hereinafter referred to as a “patient.”

In one representative embodiment, the present invention is directed to compositions that combine at least one peptide copper complex with 6-amino-1,2-dihydro-1-hydroxy-2-imino-4-piperidinopyrimidine wherein the copper peptide complex has the formula [R₁—R₂]:copper(II) or the formula [R₁—R₂—R₃]:copper(II), and wherein R₁ is an amino acid or an amino acid derivative; R₂ is histidine, arginine or a derivative thereof; and R₃ is a chemical moiety bonded to the R₂ moiety by an amide or peptide bond.

The above-mentioned compound, 6-amino-1,2-dihydro-1-hydroxy-2-imino-4-piperidinopyrimidine, is hereinafter referred to using its tradename, “Minoxidil.” It has been found that the ability of the compositions, disclosed herein, to stimulate hair growth, is greater to a surprising and unexpected extent than that of compositions that comprise at least one peptide copper complex or Minoxidil, but not both.

The disclosed compositions may be administered to areas of skin in need thereof topically or by intradermal injection. Accordingly, in additional embodiments, the disclosed compositions may further comprise a vehicle suitable for intradermal injection (e.g., sterile water), an inert and physiologically acceptable carrier or diluent, a penetration enhancement agent, a surface active agent, a sunscreen agent, a skin conditioning agent, a skin protectant, an emollient, a humectant, a hair conditioning agent, or a mixture thereof.

In another representative embodiment of the disclosed composition, the at least one peptide copper complex and/or the Minoxidil comprised therein are encapsulated in a liposome or microsponge adapted to aid in the delivery of the peptide copper complex and/or the Minoxidil to hair follicles, or to enhance the stability of the composition. The disclosed composition, in yet another embodiment, comprises Minoxidil and at least one peptide copper complex, formulated in an instrument adapted to deliver the compounds via iontophoresis to hair follicles. In a related, particular embodiment, the at least one peptide copper complex and Minoxidil are formulated for delivery to hair follicles, where the delivery is enhanced via the use of ultrasound.

In another embodiment, the composition comprises Minoxidil and at least one peptide copper complex that are formulated for topical application after a treatment, such as a laser treatment, to remove or partially remove the stratum corneum to improve the transport and delivery of the active compounds to hair follicles.

Additionally, in another embodiment, the disclosed composition may be in the form of a liquid, a cream, a suspension, a gel, an emulsion, a lotion, or an oil.

The present invention is also directed, in further representative embodiments, to methods for stimulating the growth of hair on a patient, and for arresting and reversing AGA afflicting a patient by administering to areas of the patient's skin in need thereof an effective amount of a disclosed composition, topically or via intradermal injection.

These and other aspects of this invention will be evident upon reference to the following detailed description of the invention.

### DETAILED DESCRIPTION OF THE INVENTION

As noted above, the present invention is directed to compositions that are effective for stimulating hair growth in patients so as to, for example, arrest and/or reverse AGA. Specific details of certain embodiments of the invention are set forth in the following description to provide a thorough understanding of such embodiments. One skilled in the art, however, will understand that the present invention may have additional embodiments, or may be practiced without several of the details described in the following description.

In one representative embodiment, the present invention is directed to compositions that combine at least one peptide copper complex with Minoxidil, the copper peptide complex having the formula [R₁—R₂]:copper(II) or the formula [R₁—R₂—R₃]:copper(II), wherein R₁ is an amino acid or an amino acid derivative; R₂ is histidine, arginine or a derivative thereof; and R₃ is a chemical moiety bonded to the R₂ moiety by an amide or peptide bond. Where the peptide copper complex has the formula [R₁—R₂]:copper(II), the peptide thereof is generally classified as a dipeptide.

As used herein, the expression “peptide copper complex” generally refers to a coordination compound comprising a peptide molecule and a copper(II) ion non-covalently complexed therewith. As is well understood in the art, copper (II) designates a copper ion having a valence of 2 (i.e., Cu⁺²). The peptide molecule serves as the complexing agent by donating electrons to the copper ion to yield the non-covalent complex. The peptide molecule is a chain of two or more amino acid units (or amino acid derivative units) covalently bonded together via amide linkages (for example, —CONH—), the formation of such linkages being accompanied by the elimination of water.

Generally, an amino acid consists of an amino group, a carboxyl group, a hydrogen atom, and an amino acid side-chain moiety—all bonded, in the case of an alpha-amino acid, to a single carbon atom that is referred to as an alpha-carbon. The amino acid units of the peptide copper complexes comprised in the compositions of the present invention may be provided by amino acids other than alpha-amino acids. For example, the amino acids may be beta- or gamma-amino acids, such as those shown below.

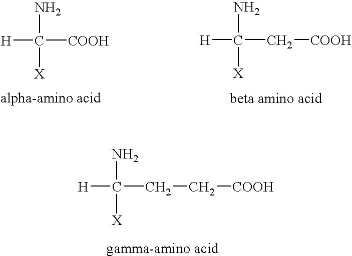

where X is the amino acid side-chain moiety bonded, along with the amino group and hydrogen, to an alpha-, beta-, or gamma-carbon atom. As is the case with proline, the amino group may be bonded to the amino acid side-chain moiety and form a ring with the alpha-, beta-, or gamma-carbon.

As another example, the amino acids include, but are not limited to, naturally occurring alpha-amino acids. Naturally occurring amino acids are those from which the amino acid units of naturally occurring proteins are derived. Some of these amino acids, along with their respective amino acid side chain moieties, are shown below in Table 1. The naturally occurring amino acids shown are all in the L configuration, referring to the optical orientation of the alpha carbon or other carbon atom bearing the amino acid side chain. A peptide molecule of the present invention may also comprise amino acids that are in the D optical configuration or a mixture of amino acids, where some are in the D optical configuration and others are in the L optical configuration.

TABLE 1
NATURALLY OCCURRING AMINO ACID SIDE-CHAIN MOIETIES

| Amino Acid Side Chain Moiety | Amino Acid |
|---|---|
| —H | Glycine |
| —CH₃ | Alanine |
| —CH(CH₃)₂ | Valine |
| —CH₂CH(CH₃)₂ | Leucine |
| —CH(CH₃)CH₂CH₃ | Isoleucine |
| —(CH₂)₄NH₃⁺ | Lysine |
| —(CH₂)₃NHC(NH₂)NH₂⁺ | Arginine |
| 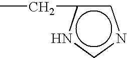 | Histidine |
| —CH₂COO— | Aspartic Acid |
| —CH₂CH₂COO— | Glutamic Acid |
| —CH₂CONH₂ | Asparagine |
| —CH₂CH₂CONH₂ | Glutamine |
| 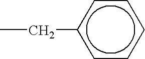 | Phenylalanine |
| 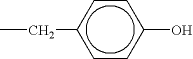 | Tyrosine |
| 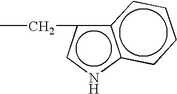 | Tryptophan |
| —CH₂SH | Cysteine |
| —CH₂CH₂SCH₃ | Methionine |
| —CH₂OH | Serine |
| —CH(OH)CH₃ | Threonine |
| 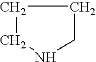 | Proline |

Other naturally occurring amino acids include hydroxyproline and gamma-carboxyglutamate.

Representative amino acid derivatives include those set forth in Table 2 below.

TABLE 2
AMINO ACID DERIVATIVES

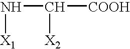

Where X₂ = H or the following moieties:
—(CH₂)ₙCH₃ where n = 1-20
—(CH₂)ₙCH(CH₃)(CH₂)ₘCH₃ where n, m = 0-20 (when n = 0,
m ≠ 0 or 1 and when n = 1, m ≠ 0)
—(CH₂)ₙNH₂ where n = 1-20 (n ≠ 4)
—(CH₂)ₙCONH₂ where n = 3-20
—(CH₂)ₙCOOH where n = 3-20

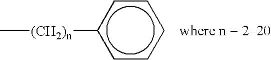

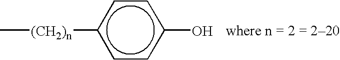

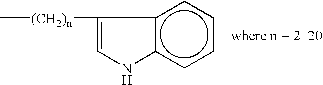

—(CH₂)ₙSH where n = 2-20
—(CH₂)ₙS(CH₂)ₘCH₃ where n, m = 1-20 (when n = 2, m ≠ 0)
—(CH₂)ₙCH₂OH where n = 1-20
—(CH₂)ₙCH(CH₃)OH where n = 1-20
And where X₁ = H or the following moieties:
—(CH₂)ₙCH₃ where n = 0-20
—(CH₂)ₙCH(CH₃)(CH₂)ₘCH₃ where n, m = 0-20

Histidine derivatives of this invention include compounds having the structure:

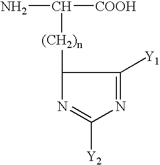

where n=1-20, and Y₁ and Y₂ are independently selected from alkyl moieties containing from 1-12 carbon atoms or an aryl moiety containing from 6-12 carbon atoms. As used herein, “alkyl” means a straight chain or branched, cyclic or noncyclic, substituted or unsubstituted, saturated or unsaturated aliphatic hydrocarbon containing from 1 to 18 carbon atoms. Representative saturated straight chain alkyls include methyl, ethyl, n-propyl and the like; while saturated branched alkyls include isopropyl, sec-butyl, isobutyl, tert-butyl, isopentyl, and the like. Representative, saturated cyclic alkyls include cyclopropyl, cyclobutyl, cyclopentyl, —CH₂cyclohexyl, and the like; while unsaturated cyclic alkyls include cyclopentenyl, cyclohexenyl, and the like. Unsaturated alkyls contain at least one double or triple bond between adjacent carbon atoms (referred to as an “alkenyl” or “alkynyl, ” respectively). Representative alkenyls include ethylenyl, 1-butenyl, isobutylenyl, 2-methyl-2-butenyl, and the like; while representative alkynyls include acetylenyl, 2-butynyl, 3-methyl-1-butynyl, and the like.

Also, as used herein, “aryl” means an aromatic carbocyclic moiety such as phenyl or naphthyl, and may be substituted or unsubstituted. “Arylalkyl,” as used herein, means an alkyl having at least one alkyl hydrogen atom replaced with a substituted or unsubstituted aryl moiety, such as benzyl (i.e., —CH₂phenyl, —(CH₂)₂phenyl, —(CH₂)₃phenyl, —CH(phenyl)₂, and the like).

In certain embodiments, n is 1, Y₂ is methyl and Y₁ is H (i.e., 3-methyl histidyl) or Y₂ is H and Y₁ is methyl (i.e., 5-methyl histidine).

Similarly, arginine derivatives of this invention include compounds having the structure:

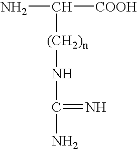

where n=1-20 (excluding n=3).

In a related embodiment, directed to compositions combining Minoxidil and a peptide copper complex having the formula [R₁—R₂—R₃]:copper(II), R₃ is at least one amino acid or amino acid derivative, as defined above, bonded to R₂ by a peptide bond (i.e., —C(═O)NH—). Where R₃ is one amino acid or amino acid derivative, then the peptide of the peptide copper complex is generally classified as a tripeptide. In another related embodiment, directed to compositions combining Minoxidil and a peptide copper complex having the formula [R₁—R₂—R₃]:copper(II), R₃ is a chemical moiety bonded to the R₂ moiety by an amide bond. The expression “chemical moiety,” as used herein and with reference to R₃, includes any chemical moiety having an amino group capable of forming an amide bond with the carboxyl terminus of R₂ (i.e., the carboxyl terminus of histidine, arginine, or derivatives thereof).

In a more particular, related embodiment where R₃ is a chemical moiety bonded to the R₂ moiety by an amide bond, R₃ is —NH₂, an alkylamino moiety having from 1-20 carbon atoms, or an arylamino moiety having from 6-20 carbon atoms. As used herein, an “alkylamino moiety” encompasses alkyl moieties containing an amino moiety, wherein the alkyl moiety is as defined above, and includes, but is not limited to, octyl amine and propyl amine. Similarly, an “arylamino moiety” encompasses aryl moieties containing an amino moiety, wherein the aryl moiety is as defined above, and includes, but is not limited to, benzylamine and benzyl-(CH₂)₁₋₁₄-amine. Further examples of suitable chemical moieties having amino groups capable of forming an amide linkage with the carboxyl terminus of R₂ include polyamines such as spermine and sperimidine.

It should be understood that R₃ may include more than one chemical moiety. For example, additional amino acids or amino acid derivatives may be bonded to the above-described peptide copper complexes comprising tripeptides to yield peptide copper complexes comprising peptides having four or more amino acids and/or amino acid derivatives. For purposes of illustration, Table 3, shown below, presents various representative examples of peptide copper complexes of the present invention.

TABLE 3
REPRESENTATIVE PEPTIDE-COPPER COMPLEXES
Examples of [R₁—R₂]: copper(II)

| | |
|---|---|
| glycyl-histidine: copper | alanyl-histidine: copper |
| glycyl-(3-methyl)histidine: copper | alanyl-(3-methyl)histidine: copper |
| glycyl-(5-methyl)histidine: copper | alanyl-(5-methyl)histidine: copper |
| glycyl-arginine: copper | alanyl-arginine: copper |
| (N-methyl)glycine-histidine: copper | (N-methyl)glycine-arginine: copper |

Examples of [R₁—R₂—R₃]: copper(II)
where R₃ is Chemical Moiety Linked by Amide Bond

| | |
|---|---|
| glycyl-histidyl-NH₂: copper | glycyl-arginyl-NH₂: copper |
| glycyl-(3-methyl)histidyl-NH₂: copper | alanyl-(3-methyl)histidyl-NH₂: copper |
| glycyl-arginyl-NH₂: copper | alanyl-arginyl-NH₂: copper |
| (N-methyl)glycine-histidyl-NH₂: copper | (N-methyl)glycine-arginyl-NH₂: copper |
| glycyl-histidyl-NHoctyl: copper | glycyl-arginyl-NHoctyl: copper |

Examples of [R₁—R₂—R₃]: copper(II)
where R₃ is Amino Acid or Amino Acid Derivative
Linked by Peptide Bond

| | |
|---|---|
| glycyl-histidyl-lysine: copper | glycyl-arginyl-lysine: copper |
| glycyl-(3-methyl)histidyl-lysine: | glycyl-(5-methyl)histidyl-lysine: |
| copper | copper |
| alanyl-histidyl-lysine: copper | alanyl-arginyl-lysine: copper |
| alanyl-(3-methyl)histidyl-lysine: | alanyl-(5-methyl)histidyl-lysine: |
| copper | copper |
| glycyl-histidyl-phenylalanine: copper | glycyl-arginyl-phenylalanine: |
| | copper |
| glycyl-(3-methyl)histidyl- | glycyl-(5-methyl)histidyl- |
| phenylalanine: copper | phenylalanine: copper |
| alanyl-histidyl-phenylalanine: copper | alanyl-arginyl-phenylalanine: |
| | copper |
| alanyl-(3-methyl)histidyl- | alanyl-(5-methyl)histidyl- |
| phenylalanine: copper | phenylalanine: copper |
| glycyl-histidyl-lysyl-phenylalanyl- | glycyl-arginyl-lysyl-phenylalanyl- |
| phenylalanyl: copper | phenylalanyl: copper |
| glycyl-(3-methyl)histidyl-lysyl- | glycyl-(5-methyl)histidyl-lysyl- |
| phenylalanyl-phenylalanyl: copper | phenylalanyl-phenylalanyl: copper |
| (N-methyl)glycyl-histidyl-lysine: | (N-methyl)glycyl-arginyl-lysine: |
| copper | copper |
| valyl-histidyl-lysine: copper | glycyl-histidyl-lysyl-prolyl- |
| | phenylalanyl- |
| prolyl-histidyl-lysine: copper | proline: copper |
| glycyl-D-histidyl-L-lysine: copper | Leucyl-histidyl-lysine: copper |
| seryl-histidyl-lysine: copper | |

Further examples of peptide copper complexes of the present invention are disclosed in U.S. Pat. Nos. 4,665,054; 4,760,051; 4,767,753; 4,810,693; 4,877,770; 5,023,237; 5,059,588; 5,118,665; 5,120,831; 5,164,367; 5,177,061; 5,214,032; 5,538,945; 5,550,183; and 6,017,888, all of which are incorporated herein by reference in their entirety.

Examples of the peptide copper complex derivatives, encompassed in embodiments of the present invention, include, but are not limited to, those disclosed and described in the above-cited U.S. Patents that are directed to peptide copper complexes, as well as those disclosed and described in the published PCT application having the international publication number WO 94/03482, incorporated herein by reference in its entirety.

The synthesis of the above-disclosed peptide copper complexes is described in detail in the above-referenced patents. For example, the peptides of the peptide copper complexes disclosed herein may be synthesized by either solution or solid phase techniques known to one skilled in the art of peptide synthesis. The general procedure involves the stepwise addition of protected amino acids to build up the desired peptide sequence. The resulting peptide may then be complexed to copper (at the desired molar ratio of peptide to copper) by dissolving the peptide in water, followed by the addition of copper chloride or other suitable copper salt and adjusting the pH to greater than 4.0.

In a yet further, more particular embodiment directed to compositions of the present invention, the ratio of peptide to copper in the peptide copper complex ranges from 1:1 to 3:1.

As noted, a composition of the present invention combines the above-described peptide copper complexes with Minoxidil. Also as previously noted, Minoxidil has also demonstrated activity as a hair growth agent, as disclosed in U.S. Pat. No. 4,596,812, incorporated herein by reference in its entirety. The synthesis of Minoxidil and similar compounds is disclosed in U.S. Pat. No. 3,461,461, also incorporated herein by reference in its entirety. Also disclosed in U.S. Pat. No. 4,596,812 is the preparation of compositions comprising Minoxidil that are suitable for topical application to skin areas in need of stimulated hair growth, as is the course and methodology of associated treatments and the results obtained thereby.

Compositions of the present invention are intended for localized application in the areas of hair loss or desirable of hair growth. Specifically, administration of the compositions of the present invention may be accomplished in any manner that will result in the delivery, including delivery to hair follicles, of an effective amount of the composition, selectively, to an area of skin (for example, the scalp) where stimulation of hair growth is desired (hereinafter, “the treatment area”). For example, administration may be accomplished by topical application directly to the treatment area, or, alternatively, by injection, such as intradermal injection, into the treatment area, including the scalp. As used herein, the expression “effective amount” means an amount of the composition that stimulates hair growth associated with the hair loss afflictions previously identified and discussed herein.

Accordingly, in one embodiment, a composition of the present invention that combines Minoxidil with at least one of the above-described peptide copper complexes, is formulated for intradermal injection to the treatment area in further comprising a vehicle suitable for such injection. Suitable vehicles include, but are not limited to: saline, bacteriostatic saline, and sterile water. The preparation of such compositions is well known in the art and described in the above-referenced patents. For example, as described therein, an amount of dried peptide copper complex, sufficient for a desired concentration thereof, is readily dissolved in water with mixing and gentle heating. Or, an aqueous solution of the desired peptide may be followed by addition of a copper salt, such as cupric chloride or cupric acetate, in the desired molar ratio to yield the desired solution of the peptide copper complex. When aqueous solutions of peptide copper complexes are prepared, the solutions are neutralized, typically with NaOH.

Also, Minoxidil is incorporated into an aqueous solution, along with at least one peptide copper complex, to yield the above-disclosed embodiment by methods well known in the art. For example, a measured amount of the active compound may be placed in a vial, the contents being sterilized and sealed therein. An accompanying vial of sterile water or aqueous peptide copper complex solution for injection is provided as a vehicle to form a dispersion prior to the administration thereof. Or, the Minoxidil can be incorporated into an aqueous solution of peptide copper complex as a pharmacologically acceptable salt thereof.

In another embodiment, the disclosed compositions combine Minoxidil and at least one of the above-described peptide copper complexes, and further comprise an inert and physiologically-acceptable carrier or diluent so as to render the compositions suitable for topical administration to the skin. Suitable inert, physiologically acceptable carriers or diluents include, but are not limited to, water, physiological saline, bacteriostatic saline (saline containing 0.9 mg/ml benzyl alcohol), creams, lotions, various types of gels, and short chain alcohols and glycols (e.g., ethyl alcohol and propylene glycol).

As an example, the use of 1% Minoxidil in equal parts of ethyl alcohol and propylene glycol for topical application to human skin to treat male pattern alopecia, is described in U.S. Pat. No. 4,596,812. Also, the use of certain disclosed peptide copper complexes in combination with inert, physiologically acceptable carriers or diluents, such as those listed above, for topical application to areas of skin to stimulate hair growth, is described in U.S. Pat. No. 5,538,945.

As noted, combining Minoxidil and at least one of the disclosed peptide copper complexes for such topical administration results in surprisingly and unexpectedly enhanced stimulation of hair growth, as shown by the examples below. Compositions adapted for topical administration, in certain embodiments, comprise at least one peptide copper complex having a concentration, based on the total weight of the composition, ranging from about 0.1% to about 20%, and further comprise Minoxidil having a concentration, based on the total weight of the composition, ranging from 0.5% to 10%.

In a related embodiment, disclosed compositions that are suitable for topical administration, further comprise a penetration enhancement agent, a surface active agent, or a mixture thereof. For example, such compositions may contain from 0.5% to 10% (by weight) of at least one surface active agent (also referred to as emulsifying agent). The surface active agent may be ionic or non-ionic.

Examples of suitable non-ionic surface active agents are nonylphenoxypolyethoxy ethanol (Nonoxynol-9), polyoxyethylene oleyl ether (Brij-97), various polyoxyethylene ethers (Tritons), and block copolymers of ethylene oxide and propylene of various molecular weights (such as Pluronic F68). Examples of suitable ionic surface active agents include sodium lauryl sulfate and similar compounds. Suitable penetration enhancing agents include dimethyl sulfoxide (DMSO), urea and substituted urea compounds. In the case of a liquid formulation for topical administration, the concentration of the penetrating enhancing agent (such as DMSO) may range from 30% to 80% of the formulation.

In another, more particular related embodiment directed to disclosed compositions, suitable for topical administration, the compositions further comprise a sunscreen agent, a skin conditioning agent, a skin protectant, an emollient, a humectant, a hair conditioning agent, or a mixture thereof.

Suitable sunscreen agents absorb, reflect, or scatter radiation in the UV range at wavelengths ranging from 290 to 400 nanometers and include, as specific examples, benzophenone-3 (oxybenzone), benzophenone-4 (sulisobenzone), benzophenone-8 (dioxybenzone), butyl methoxydibenzoylmethane (Avobenzone), DEA-methoxycinnamate (diethanolamine methoxycinnamate), ethyl dihydroxypropyl PABA (ethyl 4-[bis(hydroxypropyl)]aminobenzoate), ethylhexyl dimethyl PABA (Padimate O), ethylhexyl methoxycinnamate (octyl methoxycinnamate), ethylhexyl salicylate (octyl salicylate), homosalate, menthyl anthranilate (Meradimate), octocrylene, PABA (aminobenzoic acid), phenylbenzimidazole sulfonic acid (Ensulizole), TEA-salicylate(trolamine salicylate), titanium dioxide, and zinc oxide.

Suitable skin conditioning agents enhance the appearance of dry or damaged skin, reduce flaking, restore suppleness, and generally improve the appearance of skin. Representative examples include: acetyl cysteine, N-acetyl dihydrosphingosine, acrylates/behenyl acrylate/dimethicone acrylate copolymer, adenosine, adenosine cyclic phosphate, adenosine phosphate, adenosine triphosphate, alanine, albumen, algae extract, allantoin and derivatives, aloe barbadensis extracts, aluminum PCA, amyloglucosidase, arbutin, arginine, azulene, bromelain, buttermilk powder, butylene glycol, caffeine, calcium gluconate, capsaicin, carbocysteine, carnosine, beta-carotene, casein, catalase, cephalins, ceramides, chamomilla recutita (matricaria) flower extract, cholecalciferol, cholesteryl esters, coco-betaine, coenzyme A, corn starch modified, crystallins, cycloethoxymethicone, cysteine DNA, cytochrome C, darutoside, dextran sulfate, dimethicone copolyols, dimethylsilanol hyaluronate, DNA, elastin, elastin amino acids, epidermal growth factor, ergocalciferol, ergosterol, ethylhexyl PCA, fibronectin, folic acid, gelatin, gliadin, beta-glucan, glucose, glycine, glycogen, glycolipids, glycoproteins, glycosaminoglycans, glycosphingolipids, horseradish peroxidase, hydrogenated proteins, hydrolyzed proteins, jojoba oil, keratin, keratin amino acids, and kinetin.

Other examples of skin conditioning agents that may be used include: lactoferrin, lanosterol, lauryl PCA, lecithin, linoleic acid, linolenic acid, lipase, lysine, lysozyme, malt extract, maltodextrin, melanin, methionine, mineral salts, niacin, niacinamide, oat amino acids, oryzanol, palmitoyl hydrolyzed proteins, pancreatin, papain, PEG, pepsin, phospholipids, phytosterols, placental enzymes, placental lipids, pyridoxal 5-phosphate, quercetin, resorcinol acetate, riboflavin, RNA, saccharomyces lysate extract, silk amino acids, sphingolipids, stearamidopropyl betaine, stearyl palmitate, tocopherol, tocopheryl acetate, tocopheryl linoleate, ubiquinone, vitis vinifera (grape) seed oil, wheat amino acids, xanthan gum, and zinc gluconate.

The skin protectant refers herein to a compound that protects injured or exposed skin from harmful or irritating external compounds. Suitable examples include: algae extract, allantoin, aluminum hydroxide, aluminum sulfate, betaine, camellia sinensis leaf extract, cerebrosides, dimethicone, glucuronolactone, glycerin, kaolin, lanolin, malt extract, mineral oil, petrolatum, potassium gluconate, and talc.

The emollient refers herein to a cosmetic ingredient that can help skin maintain a soft, smooth, and pliable appearance. Emollients are able to provide these benefits, largely owing to their ability to remain on the skin surface to act as a lubricant and reduce flaking. Some examples of emollients, suitable for embodiments of this invention, are: acetyl arginine, acetylated lanolin, algae extract, apricot kernel oil PEG-6 esters, avocado oil PEG-11 esters, bis-PEG-4 dimethicone, butoxyethyl stearate, C₁₈-C₃₆ acid glycol ester, C₁₂-C₁₃ alkyl lactate, caprylyl glycol, cetyl esters, cetyl laurate, coconut oil PEG-10 esters, di-C₁₂-C₁₃ alkyl tartrate, diethyl sebacate, dihydrocholesteryl butyrate, dimethiconol, dimyristyl tartrate, disteareth-5 lauroyl glutamate, ethyl avocadate, ethylhexyl myristate, glyceryl isostearates, glyceryl oleate, hexyldecyl stearate and hexyl isostearate.

Additional examples of suitable emollients include: hydrogenated palm glycerides, hydrogenated soy glycerides, hydrogenated tallow glycerides, hydroxypropyl bisisostearamide MEA, isostearyl neopentanoate, isostearyl palmitate, isotridecyl isononanoate, laureth-2 acetate, lauryl polyglyceryl-6 cetearyl glycol ether, methyl gluceth-20 benzoate, mineral oil, myreth-3 palmitate, octyldecanol, octyldodecanol, odontella aurita oil, 2-oleamido-1,3 octadecanediol, palm glycerides, PEG avocado glycerides, PEG castor oil, PEG-22/dodecyl glycol copolymer, PEG shea butter glycerides, phytol, raffinose, stearyl citrate, sunflower seed oil glycerides, and tocopheryl glucoside.

The humectant is a cosmetic ingredient that helps maintain moisture levels in skin. Suitable examples include: acetyl arginine, algae extract, aloe barbadensis leaf extract, betaine, 2,3-butanediol, chitosan lauroyl glycinate, diglycereth-7 malate, diglycerin, diglycol guanidine succinate, erythritol, fructose, glucose, glycerin, honey, hydrolyzed wheat protein/PEG-20 acetate copolymer, hydroxypropyltrimonium hyaluronate, inositol, lactitol, maltitol, maltose, mannitol, mannose, methoxy PEG, myristamidobutyl guanidine acetate, polyglyceryl sorbitol, potassium PCA, propylene glycol, sodium PCA, sorbitol, sucrose, and urea. Other humectants may be used for embodiments of this invention, as will be appreciated by one skilled in the art.

A hair conditioning agent is a cosmetic ingredient that is used to create special effects on hair. This includes ingredients which enhance the appearance and feel of hair, increase hair body or suppleness, facilitate styling, improve gloss or sheen and improve the texture of hair that has been damaged by chemicals or environmental action. Suitable examples include: Acetylated Lanolin, Amodimethicone, Behenamidopropyl Ethyldimonium Ethosulfate, Behentrimonium Chloride, Butyrospermum Parkii (Shea Butter) Oil, Caprylic/Capric Glycerides, Ceteareth-20, Cetylpyridinium Chloride, Cocamidopropyl Betaine, Cocamidopropyl Hydroxysultaine, Cocodimonium Hydroxypropyl Hydrolyzed Keratin, Dicetyldimonium Chloride, Dimethyl Lauramine Isostearate, Glyceryl Stearate SE, Guar Hydroxypropyltrimonium Chloride, Hydrolyzed Glycosaminoglycans, Hydrolyzed Keratin, Hydroxypropyl Guar Hydroxypropyltrimonium Chloride, Isostearyl Glyceryl Pentaerythrityl Ether, Laurdimonium Hydroxypropyl Hydrolyzed Wheat Protein, Linoleamidopropyl Dimethylamine Dimer Dilinoleate, Linoleamidopropyl Ethyldimonium Ethosulfate, Linoleamidopropyl PG-Dimonium Chloride Phosphate, Methylchloroisothiazolinone, Panthenyl Hydroxypropyl Steardimonium Chloride, PEG40 Sorbitan Laurate, PEG-3 Sorbitan Stearate, PEG-6 Sorbitan Stearate, PPG-5-Ceteth-20, Propylene Glycol Dicaprylate/Dicaprate, Silk Amino Acids, Steapyrium Chloride, and Stearyl Ethylhexyldimonium Methosulfate. Other hair conditioning agents may be used for embodiments of this invention, as will be appreciated by one skilled in the art.

The present invention, in another representative embodiment, is also directed to a composition formed by combining at least one peptide copper complex with Minoxidil (“active compounds”), where the at least one peptide copper complex and/or the Minoxidil are encapsulated in liposomes or microsponges to aid in the delivery of the at least one peptide copper complex and/or the Minoxidil to hair follicles; or to increase the stability of the composition.

In yet another representative embodiment, the active compounds are formulated in an instrument adapted to deliver them to hair follicles via iontophoresis. As is appreciated by one skilled in the art, such a formulation is typically in the form of a liquid (i.e., solution), rather than a cream or gel. An example of an instrument adapted for such delivery is a large bandage comprising a chamber and delivering an electrical current. The chamber is situated so as to be in contact with the skin and comprises the formulation. In a related, particular embodiment, the active compounds are formulated for delivery to hair follicles via ultrasound. As is appreciated by one skilled in the art, ultrasound and iontophoresis enhance the delivery of the active compounds to the hair follicles by disturbing the stratum corneum, thereby improving the transport of the active compounds.

In yet another related embodiment, a disclosed composition comprises Minoxidil and at least one peptide copper complex (“active compounds”), formulated for application to the skin after a treatment, such as laser treatment, thereof. Such treatments enhance the delivery of the components of the active compounds to hair follicles by removing or partially removing the stratum corneum, thereby improving the transport of the active compounds.

In another embodiment, the disclosed composition may be in the form of a liquid, a cream, a suspension, a gel, an emulsion, a lotion, or an oil.

In another aspect, the present invention is directed to methods for stimulating the growth of hair in a patient having a hair loss affliction using the above-disclosed compositions. In one embodiment, the method comprises administering, topically or via intradermal injection, a stimulatory effective amount of a disclosed composition to an area of the patient's skin in need thereof to thereby treat a skin affliction that is AGA, M, or secondary alopecia. In a related embodiment, disclosed is a method for arresting or reversing AGA that comprises administering to the scalp of a patient in need thereof an effective amount of a disclosed composition, topically or via intradermal injection, and in a continued and periodic fashion.

For example, a disclosed method may comprise application of a disclosed composition, formulated for topical administration as describe above, directly onto the treatment area, where the application may be accomplished by rubbing the composition in the form of a lotion or gel onto the skin of the treatment area or by spraying the composition in liquid form onto the treatment area. Any quantity of the composition that is sufficient to accelerate the rate of hair growth or prevent subsequent hair loss is effective, and treatment may be repeated as often as the progress of hair growth indicates.

The following examples are provided for the purpose of illustration, not limitation.

### EXAMPLE 1

### Stimulation of Hair Growth Using a Composition Comprising at Least One Peptide Copper Complex and Minoxidil

The stimulation of hair growth using a composition that combines at least one peptide copper complex and Minoxidil (“test compounds”) was demonstrated in an experimental system incorporating radioactive cysteine in cultured hair follicles as a measure of hair growth. In this system, follicles were dissected from 4 day old mice. Healthy follicles were selected visually and cultured in serum free media supplemented with insulin, transferrin and selenium.

The activity of compositions comprising one or more of the test compounds in stimulating hair growth was measured by radioactive cysteine labeling of the newly formed hair proteins and expressed as a percentage of the activity of a control (i.e., no test compounds present). The results are shown in the table below.

| | | % of Cysteine |
|---|---|---|
| | | Incorporation |
| | | of Control |
| Test Compound | Concentration | (no addition) |
| Minoxidil | 1 mM | 119% |
| Glycyl-L-Histidyl-L-Lysyl-L-Valyl-L- | 10 μM | 113% |
| Phenylalanyl-L-Valine: Copper | | |
| Complex | | |
| Combination of | | |
| Glycyl-L-Histidyl-L-Lysyl-L-Valyl-L- | 10 μM | 127% |
| Phenylalanyl-L-Valine: Copper | | |
| Complex and | | |
| Minoxidil | 1 mM | |

The above-shown activity in stimulating hair growth is typical of the results with this model system. At the concentrations used, the Minoxidil and the peptide copper complex (Glycyl-L-Histidyl-L-Lysyl-L-Valyl-L-Phenylalanyl-L-Valine: Copper) stimulated radioactive cysteine incorporation above that of the control, while the combination thereof produced a significantly greater increase.

### EXAMPLE 2

The following are examples of suitable topical formulations that are embodiments of the compositions of the present invention. Indicated concentrations are expressed as a percentage of the total weight of the formulation.

Preparation A:

At Least One Disclosed Peptide

| | |
|---|---|
| Copper Complex | 1.0% |
| Minoxidil | 2.0% |
| Hydroxy ethyl cellulose | 3.0% |
| Propylene Glycol | 20.0% |
| Nonoxynol-9 | 3.0% |
| Benzyl Alcohol | 2.0% |
| Aqueous Phosphate Buffer (0.2 N) | 69.0% |

Preparation B:

At Least One Disclosed Peptide

| | |
|---|---|
| Copper Complex | 1.0% |
| Minoxidil | 2.0% |
| Nonoxynol-9 | 3.0% |
| Ethyl Alcohol | 94.0% |

Preparation C:

At Least One Disclosed Peptide

| | |
|---|---|
| Copper Complex | 2.0% |
| Minoxidil | 5.0% |
| Ethyl Alcohol | 45.5% |
| Isopropyl Alcohol | 4.0% |
| Propylene Glycol | 20.0% |
| Laoneth-4 | 1.0% |
| Water | 22.5% |

Preparation D:

At Least One Disclosed Peptide

| | |
|---|---|
| Copper Complex | 5.0% |
| Minoxidil | 5.0% |
| Sterile Water | 90.0% |

Preparation E:

At Least One Disclosed Peptide

| | |
|---|---|
| Copper Complex | 2.5% |
| Minoxidil | 2.0% |
| Hydroxypropyl Cellulose | 2.0% |
| Glycerine | 20.0% |
| Nonoxynol-9 | 3.0% |
| Sterile Water | 70.5% |

Preparation F:

At Least One Disclosed Peptide

| | |
|---|---|
| Copper Complex | 0.5% |
| Minoxidil | 5.0% |
| Sterile Water | 16.5% |
| Propylene Glycol | 45.0% |
| Ethanol | 30.0% |
| Nonoxynol-9 | 3.0% |

Preparation G:

At Least One Disclosed Peptide

| | |
|---|---|
| Copper Complex | 5.0% |
| Sterile Water | 10.0% |
| Hydroxypropyl Methylcellulose | 2.0% |
| Propylene Glycol | 30.0% |
| Ethanol | 50.0% |
| Nonoxynol-9 | 3.0% |

All of the U.S. patents, U.S. patent application publications, U.S. patent applications, foreign patent applications and non-patent publications listed in the Application Data Sheet, are incorporated herein by reference in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A composition comprising at least one peptide copper complex and 6-amino-1,2-dihydro-1-hydroxy-2-imino4-piperidinopyrimidine, wherein the at least one peptide copper complex has the formula:
[R₁—R₂]:copper(II)
or the formula:
[R₁—R₂—R₃]:copper(II)
and wherein R₁ is an amino acid or an amino acid derivative; R₂ is histidine, arginine or a derivative thereof; and R₃ is a chemical moiety bonded to the R₂ moiety by an amide or peptide bond.

2. The composition of claim 1 wherein the at least one peptide copper complex has the formula:
[R₁—R₂]:copper(II)
wherein R₁ is an amino acid or an amino acid derivative, and R₂ is histidine, arginine or a derivative thereof.

3. The composition of claim 1 wherein the at least one peptide copper complex has the formula:
[R₁—R₂—R₃]:copper(II)
wherein R₁ is an amino acid or an amino acid derivative; R₂ is histidine, arginine or a derivative thereof; and R₃ is a chemical moiety bonded to the R₂ moiety by an amide or peptide bond.

4. The composition of claim 3 wherein R₃ is at least one amino acid or amino acid derivative bonded to R₂ by a peptide bond.

5. The composition of claim 3 wherein R₃ is a chemical moiety bonded to R₂ by an amide bond.

6. The composition of claim 5 wherein R₃ is —NH₂, an alkylamino moiety having from 1-20 carbon atoms, or an arylamino moiety having from 6-20 carbon atoms.

7. The composition of claim 1 wherein the ratio of peptide to copper in the at least one peptide copper complex ranges from 1:1 to 3:1.

8. The composition of claim 1, further comprising a vehicle rendering the composition suitable for intradermal injection.

9. The composition of claim 1, further comprising an inert and physiologically-acceptable carrier or diluent.

10. The composition of claim 9, further comprising a penetration enhancement agent, a surface active agent, or a mixture thereof.

11. The composition of claim 9, further comprising a sunscreen agent, a skin conditioning agent, a skin protectant, an emollient, a humectant, a hair conditioning agent, or a mixture thereof.

12. The composition of claim 1 wherein the at least one peptide copper complex and 6-amino-1,2-dihydro-1-hydroxy-2-imino-4-piperidinopyrimidine are encapsulated in a liposome or microsponge adapted to aid in their delivery to hair follicles of a patient, or to enhance the stability of the composition.

13. The composition of claim 1 wherein the at least one peptide copper complex and 6-amino-1,2-dihydro-1-hydroxy-2-imino-4-piperidinopyrimidine are formulated in an instrument adapted to effect their delivery to hair follicles of a patient via inotophoresis.

14. The composition of claim 1 wherein the 6-amino-1,2-dihydro-1-hydroxy-2-imino-4-piperidinopyrimidine and the at least one peptide copper complex are formulated for delivery to hair follicles of a patient, where the delivery is enhanced by ultrasound.

15. The composition of claim 1 wherein the 6-amino-1,2-dihydro-1-hydroxy-2-imino-4-piperidinopyrimidine and the at least one peptide copper complex are formulated for application to the skin after a treatment to remove or partially remove the stratum corneum thereof.

16. The composition of claim 1 wherein the composition is in the form of a liquid, a cream, a suspension, a gel, an emulsion, a lotion, or an oil.

17. A method for stimulating the growth of hair in a patient, comprising topically, or via intradermal injection, administering to areas of the patient's skin in need thereof a stimulatory effective amount of the composition of claim 1.

18. The method of claim 17 wherein the patient has a hair-loss affliction that is androgenetic alopecia, alopecia areata, or secondary alopecia.

19. A method for arresting and reversing androgenetic alopecia comprising topically, or via intradermal injection, administering to the scalp of a patient in need thereof, in a continued and periodic fashion, an effective amount of the composition of claim 1.
